(19) United States
(12) Patent Application Publication
(10) Pub. No.: US 2015/0273121 A1
(43) Pub. Date: Oct. 1, 2015

(54) SUCTION CLEANING DEVICE

(71) Applicants: William Olivero, Champaign, IL (US); Huan Wang, Champaign, IL (US); John Georgiadis, Champaign, IL (US)

(72) Inventors: William Olivero, Champaign, IL (US); Huan Wang, Champaign, IL (US); John Georgiadis, Champaign, IL (US)

(21) Appl. No.: 14/669,432

(22) Filed: Mar. 26, 2015

Related U.S. Application Data
(60) Provisional application No. 61/970,504, filed on Mar. 26, 2014.

(51) Int. Cl. A61M 1/00

(52) U.S. Cl. CPC A61M 1/0035; A61M 2202/0014; A61M 2206/10

(57) ABSTRACT

The present disclosure provides a suction cleaning device that may be disposed between a suction wand and a suction conduit of a surgical suction system. The device assembly includes a housing having a first end, a body portion, and a second end, with at least one conduit extending there through. The body includes a valve that may move between an open and closed position. In the open position, the device operates to enable the suction path. In the closed position, the valve connects a positive pressure source to the first end to effectively remove any occlusion from the suction wand.

### CROSS-REFERENCE TO RELATED APPLICATIONS

This application incorporates by reference and claims the benefit of priority to U.S. Provisional Application 61/970,504 filed on Mar. 26, 2014.

### BACKGROUND OF THE INVENTION

The present subject matter relates generally to medical suction cleaning devices. More specifically, the present invention is a cleaning device that may be used to clear obstructive material from the end of a surgical suction instrument.

Suction devices are used in medical settings to remove objects and fluids from a body for a variety of reasons. In an operating room, for example, tissue fragments, blood, and other secretions are often carried off to improve surgical site visibility. The devices commonly employed for this purpose are sometimes referred to as “surgical suction systems.”

In its simplest form, a surgical suction system includes three components: a vacuum system, a suction conduit, and a suction wand. Such vacuum systems typically have motors appropriate for the task of surgical suction and are well known in the art. The suction conduit is an enclosed channel designed to facilitate material transfer into the vacuum system from the suction wand. The suction wand may be comprised of a handle, a tube, and a suction tip. A suction path extends from the suction wand, through the conduit, and into the vacuum system where evacuated material is deposited.

However, the nature of the evacuated material is such that that the surgical suckers frequently become occluded, usually with tissue debris. This almost always occurs at the sucker tip, which is the surgical sucker portion that typically first comes into contact with the body. A clogged sucker tip impairs use of the surgical sucker, and therefore it must be cleared for normal use to resume.

Some traditional methods for removal of the obstructing debris require a surgeon or assistant to first disconnect the suction wand from the suction conduit and then provide the suction wand to a scrub technician for cleaning. Once the tissue has been removed, the suction wand must be reattached to the suction conduit so utilization of the surgical suction system may resume. These steps comprise a time-consuming process employed in a setting where time is precious. Additionally, this process may compromise wound site sterility.

Other means of suction wand cleaning have been proposed. Herbert U.S. Pat. No. 5,770,649 discloses a surgical suction wand having a filter that may be removed, cleaned, and replaced for further use of the wand. Although Herbert describes a tissue filtering means to prevent suction wand clogging, it proposes to do so without obviating the need for the surgical sucker disassembly required to clean the device.

Prusmack U.S. Pub. No. 2007/0213667 discloses a surgical suction cleaning device configured to provide pressurized fluid used to unclog the device if it becomes occluded with tissue. Additionally, Stiehl U.S. Pub. No. 2011/0112515 discloses a gas conduit disposed within a suction handle through which carbon dioxide gas may be caused to flow toward and out the suction handle for the purpose of dislodging debris trapped at a suction tip. Although both of these publications describe suction wand cleaning means that do not require surgical suction disassembly, both would also require expensive replacement of existing equipment. Therefore, neither Prusmack nor Stiehl resolve a problem endemic to healthcare institutions—reconciliation of diminished resources and rising costs.

As shown, surgical suction system cleaning remains an inefficient or costly endeavor using known technology. Accordingly, there is a need for a device and method adapted to provide a time-efficient means for surgical sucker cleaning that may be retrofit to existing technology, as described herein.

### BRIEF SUMMARY OF THE INVENTION

In order to meet the needs described above, the present disclosure provides a suction cleaning device that may be retrofitted to existing surgical suction technology and actuated while the surgical suction system is fully assembled.

In some embodiments, the invention is directed to a device that may be disposed between a suction wand and a suction conduit of a surgical suction system. The device assembly includes a housing having a first end, a body portion, and a second end, with at least one conduit extending there through. For the purposes of the subject matter disclosed herein, the portion of the suction cleaning device that faces the suction wand will be referred to as the first end, and the portion of the suction cleaning device that faces the suction conduit will be referred to as the second end.

The first end defines the distal end of the device passageway. It is connectable to the proximal end of the suction wand. Additionally, the first end is adapted to receive suction wand ends having varying diameters and material compositions. A variety of mechanisms may be employed to connect the suction wand and the first end, including universal conduit connectors.

The body portion includes a chamber within which a first conduit and a second conduit are disposed. The first conduit is adapted to communicate at its distal end (the first end) with the suction wand and the second conduit, at its proximal end (the second end), is adapted to communicate with the suction conduit. The body includes a port conduit adapted to communicate with a pressurized fluid source.

In some embodiments, the pressurized fluid source is a pressurized saline source and partially disposed in the suction cleaning device body. However, it is contemplated that pressurized air or other fluid may be used instead of (or in addition to) the pressurized saline used as an example throughout this disclosure. Contrary to the negative pressure suction path of a surgical suction system conduit that ultimately leads to a vacuum source, here saline flows under positive pressure through the saline conduit and toward the tip of the sucker wand. A valve is used to control flow from the saline conduit through the body passageway.

The second end defines the proximal end of the passageway. It is connectable to the distal end of the sucker conduit. Like the first end, the second end is adapted to receive sucker conduit ends having varying diameters and material compositions. A variety of means may be employed to connect the suction conduit and the second end, including universal conduit connectors.

An example of a suction cleaning device includes a housing comprised of a first end, a body portion, and a second end, with at least one valve controlled conduit extending there through. The device is intended to be placed between the suction wand and suction conduit portions of a typical surgical suction system. For example, the first end may be connected to a standard suction wand and the second end may be connected to a standard suction conduit. The body portion joins both the first end and second ends and includes a valve seated therein. A first conduit passes from the first end, through the body portion, and terminates in fluid connection with the valve. Second and third passageways emanate from the valve, which alternatively controls fluid connection between the first and second passageways, or the first and third passageways. The second conduit proceeds toward the second end, and when the valve is in a closed position the first and second passageways are fluidly connected, essentially functioning as a congruous extension of the first conduit and forming a negative pressure suction path with the attached surgical sucker. The third conduit extends out of the body portion and, in a preferred embodiment, connects to a pressurized saline source. When the valve is opened to fluidly connect the first and third passageways, the negative pressure suction path is occluded and the saline is forced under positive pressure through the third passageway, through the first passageway, through the sucker wand, and ultimately out the sucker tip. Once the valve is returned to its original position, the saline fluid path is occluded and the suction path from the suction conduit to the suction wand is restored.

A method of cleaning a surgical suction system may include the steps of: inserting a suction cleaning device in fluid communication between a suction wand and a suction conduit, the suction cleaning device including a first end for securing to the suction wand, a second end for securing to the suction conduit, and a valve controlling the flow of fluid from a saline source through the suction wand and controlling the vacuum pressure through the suction conduit to the suction wand; and actuating the valve to alternatively control suction and saline flow through the device, for example closing the valve to provide negative pressure flow through the device that sucks material into the suction conduit and ultimately a vacuum source and opening the valve to provide positive pressure flow through the device that forces pressurized saline into the suction wand. The method may further include the step of using the pressurized saline to dislodge obstructive material from a tip of the suction wand.

In an embodiment, the suction cleaning device adapted to connect to a suction wand, wherein the device comprises a tubular body including a first end and a second end, wherein the first end includes a first connector configured to receive a suction wand, wherein the second end includes a second connector configured to receive a suction conduit. A first conduit extends from the first connector of the first end, and a second conduit extends from the second connector of the second end. The device also includes a port conduit extending from a port opening, wherein the port opening is located on a surface of the tubular body, wherein the port conduit is configured to receive a positive pressure source.

In addition, the device includes a value positioned between the first conduit and the second conduit, wherein the value is movable between an open position and a closed position. When the valve is in the open position, the first conduit is in fluid communication with the second conduit. When the valve is in the closed position, the port conduit is in fluid communication with the first conduit. In an example, when the valve is in the open position, the port conduit is not in fluid communication with the first conduit.

In an example, the port opening is on a side surface of the tubular body. The tubular body may be cuboidal or cylindrical.

The valve may include an actuator, wherein the actuator is operable to move the valve from the first position to the second position. The valve may be a ball valve.

The port conduit may be in fluid communication with a positive pressure liquid source. The positive pressure liquid source may include saline or water.

An advantage of the suction cleaning device is that it allows for the removal of obstructive material from a surgical suction wand without requiring disassembly of a surgical suction system.

Another advantage of the suction cleaning device is that it may be retrofit to existing surgical system technology.

Another advantage of the suction cleaning device is that it is simple, lightweight, and easy to use.

Yet another advantage of the suction cleaning device is that it may use pressurized flow of saline, a fluid commonly found in hospitals.

Additional objects, advantages and novel features of the examples will be set forth in part in the description which follows, and in part will become apparent to those skilled in the art upon examination of the following description and the accompanying drawings or may be learned by production or operation of the examples.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a perspective view of a suction cleaning device.

FIG. 2 is cross-section of the suction cleaning device showing a valve in an open position.

FIG. 3 is a cross-section of the suction cleaning device showing the valve in a closed position.

### DETAILED DESCRIPTION OF THE INVENTION

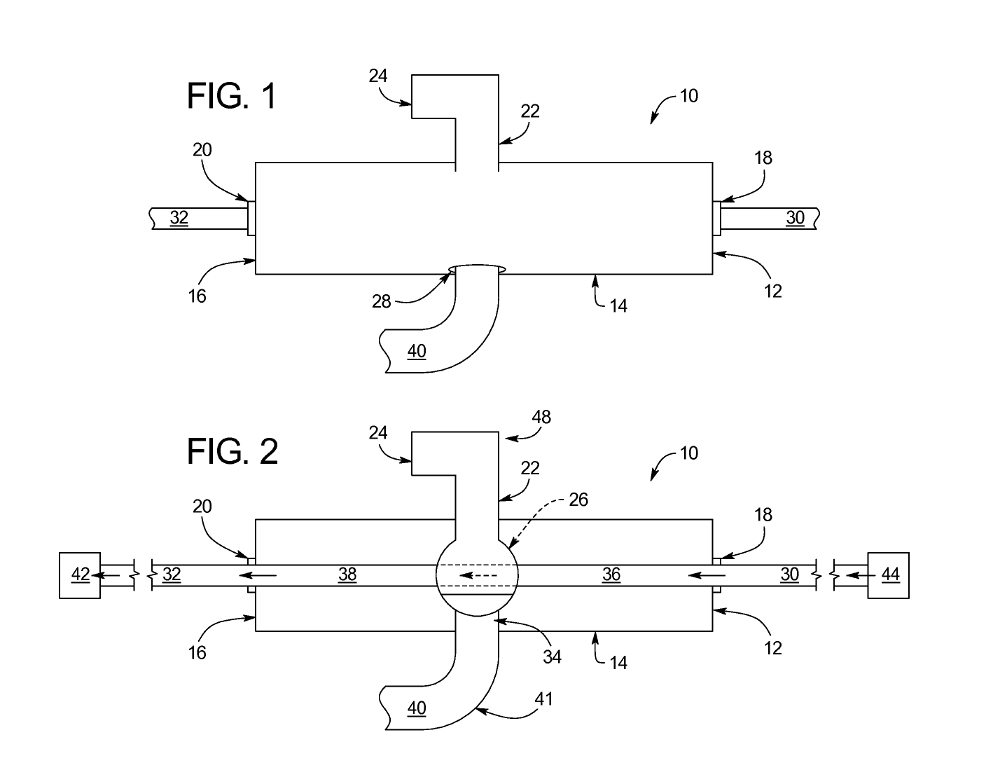

FIG. 1 illustrates an example of a suction cleaning device 10. As shown in FIG. 1, the suction cleaning device 10 includes a first end 12, a body 14, and a second end 16. The body 14 shown in FIG. 1 is formed from a polymer. However, it is contemplated that the suction cleaning device 10 may be formed from any structural material suited for use with a surgical suction device, such as stainless steel. Additionally, because the suction cleaning device 10 is intended for use in hospitals settings, it may be formed with materials that can be easily cleaned and sterilized, if needed.

In the example shown in FIG. 1, the body 14 of the suction cleaning device 10 is generally shaped as a cuboid. However, it is contemplated that the body 14 of the suction cleaning device 10 may be shaped as a cylinder, or any other shape suitable for use with a surgical suction systems.

In the example shown in FIG. 1, a first connector 18 is provided at the first end 12 of the suction cleaning device 10 for mating with a suction wand 30. The first connector 18 enables the suction wand 30 to attach to the first end 12 with a fluid tight seal. Similarly, as also shown in FIG. 1, a second connector 20 is provided at the second end 16 of the suction cleaning device 10 for mating with a suction conduit 32, enabling the suction conduit 32 to attach to the second end 16 with a fluid tight seal. Both the first connector 18 and the second connector 20 may vary in size, shape, and other configuration to appropriately mate with the objects to be secured thereto, as will be recognized by one skilled in the art based on the disclosures provided herein.

As further shown in FIG. 1, the embodiment of the suction cleaning device 10 depicted includes a valve 22, partially disposed in the body 14. In the example shown in FIG. 1, the valve 22 includes a valve actuator 24 that enables operation of the valve 22 between an open position 48 and a closed position 50. Additionally, in the examples shown in FIGS. 1-3, the valve 22 is a ball valve, though it is understood that other suitable valves may be used, as will be recognized by one skilled in the art based on the disclosures provided herein. Further, it is contemplated that more than one valve 22 may be used in the suction cleaning device 10. For example, a first valve may be used to control the flow of pressurized saline through the suction wand 30 and a second valve may be used to control the negative pressure through the suction wand 30.

As further shown in FIG. 1, the embodiment of the suction cleaning device 10 depicted includes a port opening 28 disposed in the body 14. In the example shown in FIG. 1, a port conduit 34 extends from a port opening 28, wherein the port conduit extends into the body 14 towards the valve 22. As will be recognized by one skilled in the art based on the disclosures provided herein, the port opening 28 may vary in size, shape, and other configuration to appropriately receive a pressurized conduit 40 in fluid communication with the port conduit 34. In an example, the pressurized conduit 40 includes a pressurized fluid source tube 41, wherein the pressurized fluid source tube is configured to attach to a pressurized fluid source 46. The pressurized fluid source 46 could be a pressurized fluid bag, an electrical fluid pump, a mechanical fluid pump, or any other electrical or mechanical device that may provide pressurized fluid through the pressurized conduit 40.

Turning now to FIG. 2, the body 14 of the suction cleaning device 10 includes a first conduit 36 extending from the first end 12 to a valve body 26. In the example shown in FIG. 2, the first conduit 36, in conjunction with the first connector 18 as described above, is used to fluidly connect a conventional suction wand 30 to the valve body 26. Similarly, and as also shown in FIG. 2, the suction cleaning device 10 includes a second conduit 38 extending from the valve body 26 to the second end 16. In the example shown in FIG. 2, the second conduit 38, in conjunction with the second connector 20 as described above, is used to fluidly connect a conventional suction conduit 32 to the valve body 26. FIG. 2 also depicts a port conduit 34 extending from the port opening 28, wherein the port conduit 34 is in fluid communication with the valve body 26.

As explained, the valve body 26 is a hub terminal for each of the first conduit 36, the second conduit 38, and the port conduit 40. In the example shown in FIG. 2, the valve actuator 24 placed the valve body 26 in an open position 48. The open position 48 permits a fluid connection between the first conduit 36 and the second conduit 38, while concurrently occluding the port conduit 40.

In one embodiment, the fluid connection formed between the first conduit 36 and the second conduit 38 terminates in a vacuum source 42. As such, a negative pressure suction path is formed, extending from the suction wand 30, through the suction cleaning device 10, and through the suction conduit 32 to the vacuum source 42. This suction path is depicted by a series of arrows as shown in FIG. 2, which also indicates the directional flow of detritus and effluvia 44 into the device.

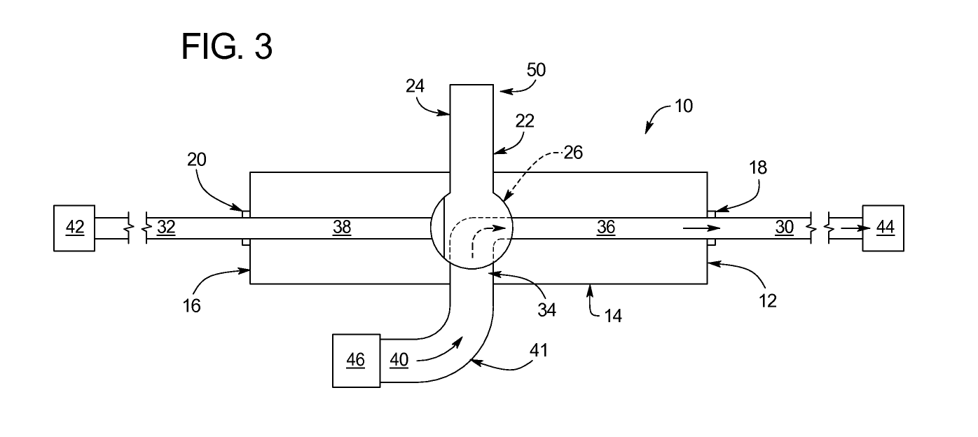

Turning now to FIG. 3, the valve actuator 24 positions the valve body 26 to be in a closed position 50. The closed position 50 permits a fluid connection between the first conduit 36 and the port conduit 40, while concurrently occluding the second conduit 38.

As described above, the port conduit 34 is connected to a pressurized fluid source 46. As such, a positive pressure fluid path is formed, extending from the pressurized fluid source 46, through the suction cleaning device 10, and out of the suction wand 30. This positive pressure fluid path is depicted by a series of arrows as shown in FIG. 3, which also indicates the directional flow of detritus and effluvia 44 away from the device.

In a preferred embodiment, the pressurized fluid source 46 provides a pressurized flow of saline or water. It is contemplated, however, that the fluid source 46 may provide any low viscosity fluid, including gases or liquids, capable of flowing through a surgical suction device.

It should be noted that various changes and modifications to the embodiments described herein will be apparent to those skilled in the art. Such changes and modifications may be made without departing from the spirit and scope of the present invention and without diminishing its attendant advantages. For example, various embodiments of the systems and methods may be provided based on various combinations of the features and functions from the subject matter provided herein.

We claim:

1. A suction cleaning device adapted to connect to a suction wand, wherein the device comprises:
a tubular body including a first end and a second end, wherein the first end includes a first connector configured to receive a suction wand, wherein the second end includes a second connector configured to receive a suction conduit,
wherein a first conduit extends from the first connector of the first end, wherein a second conduit extends from the second connector of the second end;
a port conduit extending from a port opening, wherein the port opening is located on a surface of the tubular body, wherein the port conduit is configured to receive a positive pressure source; and
a value positioned between the first conduit and the second conduit, wherein the value is movable between an open position and a closed position,
wherein, when the valve is in the open position, the first conduit is in fluid communication with the second conduit,
wherein, when the valve is in the closed position, the port conduit is in fluid communication with the first conduit.

2. The suction cleaning device of claim 1, wherein the port opening is on a side surface of the tubular body.

3. The suction cleaning device of claim 1, wherein the tubular body is cuboidal.

4. The suction cleaning device of claim 1, wherein the tubular body is cylindrical.

5. The suction cleaning device of claim 1, wherein the valve includes an actuator, wherein the actuator is operable to move the valve from the first position to the second position.

6. The suction cleaning device of claim 7, wherein the valve is a ball valve.

7. The suction cleaning device of claim 1, wherein, when the valve is in the open position, the port conduit is not in fluid communication with the first conduit.

8. The suction cleaning device of claim 1, wherein the port conduit is in fluid communication with a positive pressure liquid source.

9. The suction cleaning device of claim 7, wherein the positive pressure liquid source includes saline.

10. The suction cleaning device of claim 7, wherein the positive pressure liquid source includes water.

11. A suction cleaning device adapted to connect to a suction wand, wherein the device consists of:
a tubular body including a first end and a second end, wherein the first end includes a first connector configured to receive a suction wand, wherein the second end includes a second connector configured to receive a suction conduit,
wherein a first conduit extends from the first connector of the first end, wherein a second conduit extends from the second connector of the second end;
a port conduit extending from a port opening, wherein the port opening is located on a surface of the tubular body, wherein the port conduit is configured to receive a positive pressure source; and
a value positioned between the first conduit and the second conduit, wherein the value is movable between an open position and a closed position,
wherein, when the valve is in the open position, the first conduit is in fluid communication with the second conduit,
wherein, when the valve is in the closed position, the port conduit is in fluid communication with the first conduit.
